(19) United States
(12) Patent Application Publication
(10) Pub. No.: US 2003/0165580 A1
(43) Pub. Date: Sep. 4, 2003

(54) Safe pharmaceutical composition for treatment and prevention of gynecological disease

(76) Inventor: Xinxian Zhao, Shenzhen (CN)

Correspondence Address: Xinxian Zhao, 67-08 168th Street, Flushing, NY, 11365, US

(21) Appl. No.: 10/087,073

(22) Filed: Mar. 4, 2002

(51) Int. Cl. A61K 35/78

(52) U.S. Cl. 424/725

(57) ABSTRACT

The present invention relates to a new and safe pharmaceutical composition for treating and preventing gynecological disease and increasing immune function.

Specifically, this invention provides a new and safe composition, which is ligustilide (LIG) extracted from Dang Gui. The chemical structure of ligustilide is indicated.

### BACKGROUND OF THE INVENTION

This invention relates to a new and safe pharmaceutical composition for treatment and prevention of gynecological disease and increase immune function. Specifically, this invention provides a new and safe composition, which is Ligustilide (LIG) extracted from Dang Gui. The structure of Dang Gui is indicated below.

### DESCRIPTION OF PRIOR ART

Many of the most common medical problems experienced by women are related to their hormone disorders. Cynecological disorders include the following common symptoms: lower abdominal pain, external genitalia pain, low back pain and pelvic pressure, abnormal bleeding, leukorrheagia and amenorrhea. Dang Gui has been already used for treating and preventing gynecological disease including above symptoms long time. In clinical, hormone is used for treating gynecological disease too. However, Hormone treatment, has an obviously side effect. But Dang Gui has no side effect.

Estrogens (ES) are a typical example. ES have been used extensively for replacement therapy in estrogen-deficient patients. The estrogen deficiency may be due to primary failure of development of the ovaries, castration, or menopause. ES are widely used after the menopause. The need for and response to estrogen are quite variable, and many symptoms and disorders in menopausal women are probably unrelated to its deficiency. ES combined with progestins can be used to suppress ovulation in patients with intractable dysmenorrhea or when suppression of ovarian function is used in the treatment of hirsutism and amenorrhea due to excessive secretion of androgens by the ovary.

A large number of adverse effects have been reported with the therapeutic use of ES. Many other effects reported in conjunction with hormonal contraceptives may be related to their estrogen content.

Estrogen therapy has now become the major cause of postmenopausal bleeding. Unfortunately, vaginal bleeding at this time of life may be due to carcinoma of the endometrium, and a large number of women have been and will be subjected to dilatation and curettage of the uterus unnecessarily.

A brief summary of some side effects is as follows: Mausea, breast tenderness, edema, chloasma: Decrease estrogen content, early and midcycle spotting, decreased flow or amnorrhea, weight gain, hair growth, depression, tiredness: Decrease progestion content, excessive bleeding.

Late cycle spotting including: nausea, mastalgia, breakthrough bleeding, and edema are related to the amount of estrogen in the preparation. They were more common with the sequential preparations because of the large amounts of estrogen present and can often be alleviated by a shift to a combination agent or to a preparation containing smaller amounts of estrogen.

Headache is mild and often transient. Migraine is often made worse and has been reported to be associated with an increased frequency of cerebrovascular accidents.

Breakthrough bleeding is the most common problem of estrogen. Weight gain is more common with the combination agents containing androgenlike progestins. Increased skin pigmentation may be distressing in dark-skinned women. It tends to increase with time, the incidence being about 5% at the end of the first year and about 40% after 8 years. Acne may be exacerbated. Hirsutism may also be aggravated. Vaginal infections are more common and more difficult to treat in patients who are receiving oral estrogen Also thromboembolism was one of the earliest of the serious unanticipated effects.

If estrogens used longer time, the following side effects will be caused. Venous thromboembolic disease—The risk for this disorder is increased during the first month of contraceptive use and remains constant for several years or more. The risk returns to normal within a month when use is discontinued. The risk of venous thrombosis or pulmonary embolism among women with predisposing conditions may be higher than that in normal women. The incidence of this complication is related to the estrogen content of oral contraceptives. The risk of superficial or deep thromboembolic disease in patients treated with oral contraceptives is not related to age, parity, mild obesity. Decreased venous blood flow, endothelial proliferation in veins and arteries, and increased coagulability of blood resulting from changes in platelet coagulation and fibrinolytic systems contribute to the increased incidence of thrombosis. The major plasma inhibitor of thrombin, antithrombin 3, is substantially decreased during oral contraceptive use.

Myocardial infarction—The use of oral estrogen is associated with a higher risk of myocardial infarction in women who have a history of preeclampsia or hypertension, or who have hyperlipoproteinemia or diabetes. The association with myocardial infarction is thought to involve acceleration of atherogenesis, decreased glucose tolerance, decreased levels of high-density lipoproteins and increased levels of low-density lipoproteins, and increased platelet aggregation.

Cerebrovascular disease—The risk of strokes is concentrated in women over 35. It is increased in current users. The pattern of risk is similar to that seen for myocardial infarction. Elevations in blood pressure may also increase the risk, since there is a 3- to 6-fold increase in the incidence of overt hypertension in women taking oral estrogen. Available data indicate that oral estrogen increase the risk of various cardiovascular disorders at all ages.

Gastrointestinal disorders—These agents have also been found to increase the incidence of symptomatic gastrointestinal disorders disease. It also appears that the incidence of hepatic adenomas is increased in women taking oral estrogen. Ischemic bowel disease secondary to thrombosis of the celiac and superior and inferior mesenteric arteries and veins has also been reported in women using this estrogen.

In summary, estrogens caused a lot of side effects. Contrary, Dang Gui and LIG have no side effects as mentioned above.

Natural drugs are developed fast recently. More important is in August 2000, FDA issued the draft version of “Guidance for Industry Botanical Drug Products (“Guidance”), which explains when a botanical drug may be marketed under an OTC drug monograph and when FDA approval of a NDA is required for marketing. It strongly shows the commitments of FDA to further open U.S market to qualified botanical drugs. Many new regulatory approaches to access the safety and effectiveness of botanical drugs are recommended in the Guidance as follow, which differentiate botanical drugs from synthetic or highly purified drugs and hence remove some long-standing technical obstacles for the ultimate approval of FDA to admit qualified botanical drugs as new drugs in the US.

The development of natural drug has progressed very successfully now. Taxol, for example, is a novel anticancer plant drug isolated from the needles and bark of the western yew, Taxus brevifolia. It is the prototype for a new class of anti-tumor drugs, which are characterized by their capacity to promote the assembly of microtubules. In tissue culture, taxol is a potent inhibitor of cell replication in the G or M phase of the cell cycle. Cytoplasmic microtubules in these drug-related cells are resistant to disassembly. In addition, these cells display an unusual inter-phase microtubule cytoskeleton consisting of free and bundled microtubules and lose their ability to migrate.

Clinical studies have demonstrated taxol to have antitumor activity indlude against advanced ovarian and breast cancer. Recent clinical trials have shown that paclitaxel and docetaxel may also be useful agents for the treatment of non-small-cell lung cancer, head and neck cancer, and other types of cancer.

The promising clinical activities of the anticancer taxol combined with potential problems due to limited supply generated a multitude of programs worldwide, such as broader clinical trials and in-depth clinical evaluation of anticancer taxol, efforts to provide an adequate supply of paclitaxel for clinical trials and for cancer patients, development of better drug formulations, biochemical studies to elucidate the precise mechanism of action, chemical studies to obtain structure activity information, development of second-generation paclitaxel analogues, elaboration of semisynthetic methods for the large scale production of the anticancer taxol, and efforts directed at the total synthesis of paclitaxel. Example of taxol indicated that pure natural medicine is very important.

So far, there is no any previous art reported that safe pharmaceutical composition is used for treating and preventing gynecological disease and increasing immune function.

### DETAILED DESCRIPTION

According to this invention, ligustilide (LIG) is a safe and efficient ingredient, which has been used for treating and preventing gynecological disease and increasing immune function. LIG is extracted from an herb named Dang Gui.

The following examples are not intended, however, to limit or restrict the scope of the invention in any way, and should not be construed as providing conditions, parameters, reagents, or starting materials which must be utilized exclusively in order to practice the present invention.

### EXAMPLE 1

Extraction of Ligustilide

Ligustilide extracted from the herb of Dang Gui. Dried powder of plant was extracted with hexanol. Hexanol extraction was concentrated under reduced pressure and still residue obtained. The residue was resolved in ether. Ether solution extracted by 5% of NaOH. Ether level was collected and washed by water. Ether level was collected and recovered under reduced pressure. Oil level (1) was obtained. Oil (1) was passed through a column of silica gel and elute with chloroform (CHCl₃). Solution of CHCl₃ recovered under reduced pressure and Oil level (2) was collected. Oil level (2) was distilled and 168° C.-169° C. fraction was collected. Oil level (3) obtained. Oil level (3) washed by water. Oil level (3) was distilled again and 168° C.-169° C. fraction was collected again. The final product is ligustilide. It has the following structure.

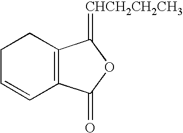

BP: 168-169° C.

n²⁵ 1.5649.

UVλ^{EtOH} nm: 215, 272, 320.

IRλₘₐₓ μ: 3.44, 5.68.

MSm/e (%): 190 (M⁺) (72), 161 (100), 133 (36), 105 (70), 77 (64).

NMR (CHCl₃) δ: 0.96, 1.49˜1.40, 2.28˜2.89, 5.28, 6.00˜6.41.

### EXAMPLE 2

LIG Injecting Preparation

LIG, according to the conventional methods, was made as ampoules or other injection preparation, then sterilized. Type XGI.S double door functional ampoule sterilizing machine is used for manufacturing of LIG injection. The function of facilities includes sterilization, leakage detection and washing. Microcomputer (PC machine) is applied in the principal controlling system. Dose is intramuscularly 5-100 mg daily.

### EXAMPLE 3

LIG Oral Preparation

LIG powder granulated accorded to the conventional granulation method. The mixture content decreased from 100% to 93%. The 7% of content was different types of fillers. Disintegrants and lubricants were used: microcrystalline cellulose (Avicel PH 105, PH 101, PH 102, PH 200, all from FMC Corp., Lehmann and Voss and Co., Hamburg, Germany; and Vivacel 200, Rettenmaier and Söhne GmbH, Ellwangen-Holzmühle, Germany), microfine cellulose (Elcema P 050, P 100, G 250, all from Degussa AG, Frandfurt, Germany; and Tablettierhilfsmittel K, Merck KGaA, Darmstadt, Germany), lactose cellulose granulate (Cellactose, Meggle, Wasserburg, Germany), α-lactose monohydrate (Lactose D 80, Meggle, Wasserburg, Germany), and modified maize starch (Starch 1500, Colorcon GmbH, Königstein, Germany).

The disintegrants tested were the following: cross-linked sodium carboxymethyl cellulose (Ac-Di-Sol, FMC Corp./Lehmann and Voss and Co.; and Nymcel ZSB 10, Nymcel ZSB 16, METSÄ-SERLA, Njimegen, Netherlands), Cross-linked calcium carboxymethyl-cellulose (ECG 505, FMC Corp./Lehmann and Voss and Co.), potato starch (Caeleo, Hilden, Germany), sodium starch glycolate (Explotab, Gustav Parmentier, Frankfurt, Germany; and Primojel, AVEBE Deutschland, Düsseldorf, Germany), cross-linked polyvinylpyrrolidone (Kollidon CL, BASF AG, Ludwigsburg, Germany; and Polyplasdone XL, ISP Deutschland, Frechen, Germany), and low-substituted hydroxypropyl-cellulose (L-HPC LH 22, L-HPC LH 31, both from Shin-Etsu Chemical Co., Ltd., Tokyo, Japan).

For lubrication, the following were used: magnesium stearate (Otto Bärlocher GmbH, Munich, Germany), glyceryl tristearate (Dynasan 118, Hüls Ag, Witten, Germany), and polyethylene glycol (PEG 6000, Hoechst AG Frankfurt/Main, Germany). Colloidal silicon dioxide (Cab-O-Sil M 5, Cabot GmbH, Hanau, Germany; Syloid 244, W. R. Grace and Co., Lexington, Ky., and Aerosil 200, Degussa AG, Frankfurt/Main, Germany) and hydrophobic colloidal silicon dioxide (Aerosil R 972, Degussa AG) were used. As a stabilizer, ascorbic acid (Merck KGaA, Darmstadt, Germany) was added.

The content of HUE was kept constant at a level of 100 mg per tablet. Tablet weight was varied between 100-105 mg. Tablet mixtures were mixed for 10 min in the Turbula mixer (type T2C, Willy Bachofen, Basel, Switzerland). The n lubricants were sieved through a 315-μm sieve into the mix. Final mixing was carried out for 5 min at 42 rpm in the Turbula mixer. The mixtures were compressed using a rotary press (Korsch PH 103, Korsch, Berlin). The lower compression roller was instrumented with four strain gauges (type 3/120 LY 11, Holtinger Baldwin, Inc., Darmstadt, Germany). A Philips carrier-frequency bridge (PR 9307 Philips, Kassel, Germany) was used for signal amplification. Each batch was compressed at different levels of compression force in the range of 1 to 25 kN. As a stabilizer, ascorbic acid (Merk KGaA, Darmstadt, Germany) was added. Sugar-coating operation was also performed conventionally.

The dosage of LIG is orally 50-200 mg daily.

### EXAMPLE 4

Effects of LIG on Gene Product of Uterine

It is known that peroxidase induction is a useful market for estrogen-effected growth responses in uterine. The uterine peroxidase is a useful gene product for assessing effect of hormones on uterine. The following experiment shows that effects of LIG on gene product of uterine.

Methods

Female Sprague-Dawley rats at 20 days of age, allowed free access to food and water. Castrate rats were the same strain. Rats of normal group were administered in saline solution by intraperitoneal injection. Rats of control group were injected cycloheximide. Rats of treatment group injected cycloherimide+LIG.

At the indicated times the rats were sacrificed by decapitation. The entire uterus from oviduct to cervix was removed, quickly dissected free of fat and connective tissue, blotted on hard filter paper, and weighed. Each uterus was minced with a scissors into a 12-ml polycarbonate tube and homogenized, 10 sec with ice cooling in 10 mM Tris.HCl, pH 7.2, buffer (25 mg of tissue per ml unless otherwise stated), with homogenizer. The homogenates were centrifuged for 45 min at 39,000×g at 2°. The hypotonic supernatant fraction contains no peroxidase activity. The peroxidase activity was solubilized by rehomogenizing the 39,000×g sediment with 10 mM Tris.HCl, pH 7.2, buffer containing 0.6 or 1.2 M NaCl, or more effectively with 0.5 M CaCl₂. The extract was collected by centrifugation for 45 min at 39,000×g at 2°.

Assays were performed. The assay mixture, 3.0 ml total volume, contained 13 mM guaiacol and 0.3 mM H₂O₂ in the extraction buffer. The reaction was started by the addition of enzyme (extract), and initial rates of guaiacol oxidation were determined from the increase in absorbance at 470 nm for the first minutes.

TABLE 1
Effects of LIG on gene product

| | Peroxidase activity |
|---|---|
| Group | Enzyme units/10 mg uterus |
| Normal (N) | 8.0 ± 0.9 |
| Control (C) | 2.4 ± 0.4 |
| Treatment (T) | 7.6 ± 0.8* |

P < 0.001 compared with control group

The data of Table 1 showed that LIG could increase peroxidase activity of uterine significantly. It means that LIG could increase normal gene product of uterine when uterine was injured.

### EXAMPLE 5

Effect of LIG on DNA Synthesis

Female Sprague-Dawley rats at 20 days of age, allowed free access to food and water and, as immature rats, used by age 23 days. Castrate rats were the same strain. Rats of normal group were administered in saline solution by intraperitoneal injection. Rats of control group were injected cycloheximide. Rats of treatment group injected cycloherimide+LIG.

At the indicated times the rats were sacrificed by decapitation. The entire uterus from oviduct to cervix was removed, quickly dissected free of fat and connective tissue, blotted on hard filter paper, and weighed. Each uterus was minced with a scissors into a 12-ml polycarbonate tube and homogenized, 10 sec with ice cooling in 10 mM Tris.HCl, pH 7.2, buffer (10 mg of tissue per ml unless otherwise stated), with homogenizer. Aliquets used for DNA analysis. Determination of DNA is used methods of ³H-TdR. The results are shown as following table.

TABLE 2
Effect of LIG on DNA synthesis

| | DNA synthesis |
|---|---|
| Group | (CPM/10 g uterine tissues) |
| Normal (N) | 1250 ± 140 |
| Control (C) | 721 ± 95 |
| Treatment (T) | 1052 ± 110 |

The data of above table indicated that LIG could increase DNA synthesis of uterine, which injured by cycloheximide.

### EXAMPLE 6

Effect of LIG on Receptor

Methods of animal and uterus preparation are as described in example 4. Receptor preparation is described as follows.

Plasma membrane isolation from uterine tissues for the determination of receptor was carried out as the following. Tissues were excised, suspended in 10 vols. (w/v) of a buffer containing 250 mM sucrose, 25 mM Tris-HCl, 1 mM EGTA (pH 7.4), and homogenized at 4° C. The crude homogenate was centrifuged to isolate the plasma membrane fraction, which was resuspended in Tris buffer and stored under liquid nitrogen for receptor analysis. Ten nm [³H]estradiol was used for measuring the estrogen (ER) and progesterone (PR) binding respectively. Specific binding was determined by subtracting the non-specific binding (radiolabeled substrate+10 μl of a 200-fold molar excess of unlabeled hormone) from total binding. Receptor levels were determined.

Binding kinetics was determined by Scatchard analysis on all treatment groups. Briefly, 50 μg of pooled uterine plasma membrane preparations were incubated with 1000, 100, 1, 0.1 and 0.01 pM radiolabeled hormone with and without 200-fold excess radioinert hormone. Data were plotted as Bound/Free vs. f mol hormone bound and K_{D} values were determined from the slopes of these plots.

TABLE 3
Effect of LIG on receptors

| | Uterine receptor levels | |
|---|---|---|
| | (molar/mg protein) | |
| Group | Estradiol | Progesterone |
| Normal (N) | 89.0 ± 13.5 | 468 ± 30.7 |
| Control (C) | 61.8 ± 5.5 | 302 ± 30.5 |
| Treatment (T) | 91.2 ± 11.5 | 439.5 ± 48.0* |

*P < 0.001 compared with control group

Above data indicated that LIG increased receptors levels of estradiol and progesterone significantly.

### EXAMPLE 7

Effect of LIG on Uterine RNA

Uterine samples (100 mg) were digested in a 4 M guanidium thiocyanate solution, to which was added 50 μl of 2 M sodium acetate (pH 4.0), 500 μl phenol, and finally 200 μl chloroform/isoamyl alcohol (24:1, v/v) sequentially, with mixing after each addition. The RNA from the resultant solution was pelleted by mixing with 1 vol. isopropanol and incubating at −20° C. for 2 h and spun at 12000×g for 12 min, then resuspended in 0.5% sodium dodecyl sulfate (SDS). The RNA suspension was incubated for 10 min at 65° C. and stored at −20° C. until use. RNA concentration was determined by absorbance intensity at 260 nm.

TABLE 4
Effect of LIG on uterine RNA

| Group | RNA units |
|---|---|
| Normal (N) | 0.89 ± 0.09 |
| Control (C) | 0.50 ± 0.06 |
| Treatment (T) | 0.92 ± 0.12* |

*P < 0.01 significantly compared with control group

The data of Table 1-4 indicated that LIG could increase uterine and hormone function of female animal.

### EXAMPLE 8

Effects of LIG on Hemopoietic System

Effects of LIG on hemopoietic system were investigated. Results showed that LIG could markedly improve the recovery rate of hemopoieses in treatment mice by cyclophosphamide.

With increased cells in bone-marrow (BMC), endogenous colonies in spleen and higher ³H-TdR uptake in marrow and spleen. The level of serum colony stimulating factor (CSF) increased after injection. It was found that LIG protected the stem cells of bone marrow in mice from the killing effect of cyclophosphamide.

Pharmacological effects as illustrated by the following table: by means of the spleen colony assay technique the action of LIG on bone marrow stem cells (CFU-S)

TABLE 5
Effects of LIG on hemopoietic system

| Group | Number of sample | Mean (CFU-S ± SD) |
|---|---|---|
| Normal (N) | 10 | 29.8 ± 3.2 |
| Control (C) | 10 | 6.9 ± 0.9 |
| Treatment (T) | 10 | 28.5 ± 3.5* |

*P < 0.01 different from control group

### EXAMPLE 9

Effect of LIG on Phagocytosis of Peritoneal Macrophage of Mice

Experimental Procedure:

Male mice weight 18-20 g were used in the experiments and were divided into treated (LIG) and control groups. The dosage of LIG was 5.5 mg/kg injected intraperitoneally. The control mice were injected with same volume of normal saline. These injections were repeated daily for 5 days, both treated and control group were injected intraperitoneally with cyclosphosphamide. The dosage of cyclophosphamide is 4.5 mg/kg.

The same experimental procedure for example 3, 4 and 5 in testing mice were used. Added 0.02 ml of 5% washed chick red blood cell suspension to 0.5 ml of the peritoneal exudates. Shook gently to mix and incubate at 37° C. for 5 minutes. Dipped two cover slips, closed to each other, in the above mixture and incubated for 30 minutes for the migration of the macrophages along the cover slips, fixed and stained with Sharma stain. Examined microscopically for: Phagocytic rate—number of macrophages with phagocytized chick red blood cells per 100 macrophages counted. Method of animal model is regular.

Results:

TABLE 6
Effect of LIG on phagocytosis of peritoneal

| Group | Number of sample | Phagocytic (rate ± SD) |
|---|---|---|
| Normal (N) | 10 | 32.5 ± 4.2 |
| Control (C) | 10 | 7.2 ± 0.9 |
| Treatment (T) | 10 | 26.8 ± 3.1* |

*P < 0.01 different from control group

### EXAMPLE 9

Effect of LIG on White Blood Cells in Rats Treated by Cyclophosphamide

Action of LIG and cyclophosphamide on white blood cells was investigated by means of white blood cells assay. It was revealed that LIG protected white blood cells in rats from the killing effect of cyclophosphamide. Method of testing in animal is standard. The dosage of LIG and cyclophosphamide is the same as in above examples. Time of treatment is 10 days. The results are listed below table:

TABLE 7
Effect of LIG on white blood cells

| | | White blood cells × |
|---|---|---|
| Group | Number of sample | 10³/cm³ ± SD |
| Normal (N) | 20 | 14.9 ± 1.8 |
| Control (C) | 20 | 6.3 ± 7.2 |
| Treatment (T) | 20 | 13.8 ± 2.0* |

*P < 0.01 different from control group

### EXAMPLE 10

Effect of LIG on Lymphoblastoid Transformation

By means of ³H-TdR liquid scintillation assay technique, the action of LIG on lymphoblastoid transformation was investigated method:

(1) Experimental procedure of animal is the same as in Example 2.

(2) Lemphoblastoid transformation test:

I. Reagents and Conditions for Cell Culture

a. Culture media—RPMI 1640, medium 199 minimal essential medium (Eagle).

b. Buffer—Hepes buffer, the final concentration at 37° C. was 25 mM, to maintain the pH of the medium at 7.31.

c. Serum—generally 15-205 fetal bovine serum was incorporated, for lymphocytes from mice, 5% was used.

d. Gaseous phase—5% CO₂ in air.

e. Cell concentration—generally 1-2×10⁶/ml.

f. Stimulants—20 μl/ml for phytohemagglutinin containing polysaccharide (PHA-M) or 10 μl/ml for polysaccharide-free purified phytohemagglutinin (PHA-P).

II. Measured by Liquid Scintillation

a. The conditions of cell culture are same as above. ³H-TdR was added after 48 hours of incubation at a final concentration of 1 μCi/ml and continued the incubation for 24 hours.

b. Washed the cells twice with cold normal saline and the erythrocytes were lysed. The intact lymphocytes were again washed once with cold saline. Spun down the lymphocytes and added 2 ml of 10% trichloroacetic acid to precipitate the protein. Washed twice with normal saline. Added 2 ml of ethanol:ether (1:1) to wash once. 0.2 ml of formic acid was then added for digestion till the precipitate was dissolved.

c. Added 4 ml of scintillation fluid to 0.1 ml of the final sample and counted in a liquid scintillation counter.

Results are listed in the following table:

TABLE 8
Effect of LIG on lymphoblastoid transformation

| Group | Number of sample | CPM ± SD |
|---|---|---|
| Normal (N) | 10 | 2050 ± 250 |
| Control (C) | 10 | 800 ± 200 |
| Treatment (T) | 10 | 1650 ± 270* |

*P < 0.01 different from control group

### EXAMPLE 11

Effect of LIG on Interleukin-2 (rIL-2)

The methods of determination rIL-2 were regular. The experimental data are listed in the following Table 9.

TABLE 9
Effect of LIG on interleukin-2 (rIL-2)

| Group | Number of sample | IL-2 (U/ml) ± SD |
|---|---|---|
| Normal (N) | 10 | 82.8 ± 9.2 |
| Control (C) | 10 | 62.0 ± 6.0 |
| Treatment (T) | 10 | 75.6 ± 8.9 |

### EXAMPLE 12

Effects of LIG on Immune Function of Human Blood Lymphocytes

Twenty (20) old volunteers (60-70 years of age) and 10 healthy young persons participated in the experiment.

2 ml of venous blood, heparinized was obtained from each of the participants. The Study of the effects of LIG was carried out by using Eagle's Minimal Essential Medium MEM). MEM was supplemented with 0.125 ml of heat-inactivated fetal calf serum, 100 units of Penicillin and 0.1 mg of streptomycin per ml of medium. Culture medium was divided into treated (LIG) and control group. LIG was added to the culture medium of treatment group. The culture medium of control group was mixed with same volume as that of LIG of normal saline on the 72 hours of culture. The ³H-thymidine (³H-TdR) was added into all the cultures (2 μci/ml) for last 12 hours of culture. The cells were harvested on 0.45 μm filters, washed with phosphate buffer (ph 7.4) and bleached with H₂O₂. The filters were then dried and the incorporation of ³H-TdR into lymphocytes cell was measured by scintillation counter.

TABLE 10
Effects of LIG on immune function of human blood lymphocytes

| Group | Number of sample | CPM |
|---|---|---|
| Normal (N) | 10 | 2580 ± 310 |
| Control (C) | 10 | 1670 ± 205 |
| Treatment (T) | 10 | 2180 ± 360 |

The data of Table 5-10 indicated that LIG could increase human immune function significantly.

### EXAMPLE 13

Pharmaceutical Preparations

Each dose for an adult is 50-500 mg. Using 50 kg as the average weight of an adult the dosage is 1-10 mg/kg. Therefore, it is very sage.

The preparation of pharmaceutical composition or drugs, which can be accomplished by the extraction methods set forth above or any conventional methods for extracting the active principles from the plants. The novelty of the present invention resides in the mixture of the active principles in the specified proportions to produce drugs, and in the preparation of dosage units in pharmaceutically acceptable dosage form. The term “pharmaceutically acceptable dosage form” as used hereinabove includes any suitable vehicle for the administration of medications known in the pharmaceutical art, including, by way of example, capsules, tablets, syrups, elixirs, and solutions for parenteral injection with specified ranges of drugs concentration.

In addition, the present invention provides novel methods for treating and preventing a variety of cancer conditions and control cancer cells with produced safe pharmaceutical agent.

It will thus be shown that there are provided compositions and methods which achieve the various objects of the invention and which are well adapted to meet the conditions of practical use.

As various possible embodiments might be made of the above invention, and as various changes might be made in the embodiments set forth above, it is to be understood that all matters herein described are to be interpreted as illustrative and not in a limiting sense.

What is claimed as new and desired to be protected by Letter Patent is set forth in the appended claims:

1. A safe pharmaceutical composition for treating and preventing gynecological disease and increasing immune function comprises Ligustilide (LIG) and its derivate.

2. A safe pharmaceutical composition of claim 1 wherein the amount to sufficient to treat gynecological disease and increase immune function is about 25-300 mg daily of LIG.

3. A safe pharmaceutical composition of claim 1, which is tablet or capsule form.

4. A dosage unit according to claim 1 wherein said dosage form is tablet which includes in addition pharmaceutical acceptable binder and excipients.

5. A dosage unit according to claim 1, wherein said dosage form is a solution for parenteral injection which includes in addition a liquid vehicle suitable for parenteral administration.

6. A process for producing Ligustilide (LIG), which used for treating and preventing gynecological disease and increasing immune function comprises:
a. Extracting said powder of Dang Gui with 95% of ethanol;
b. The hexanol extraction was recovered and the residue obtained;
c. The residue was resolved in ether;
d. The ether extracted by 5% of NaOH;
e. Ether was collected and washed by water;
f. Ether was passed through a column of silica gel and elute with CHCl₃;
g. Solution of CHCl₃ recovered under reduced pressure and oil was collected;
h. Oil was distilled and 168° C.-169° C. fraction was collected;
i. Oil washed by water and distilled;
j. 168° C.-169° C. fraction was collected again; and
k. The final product is ligustilide.

7. A safe pharmaceutical composition for treating and preventing gynecological disease and increasing immune function comprises Ligustilide (LIG), which extracted from an herb named Dang Gui.
